(19) United States
(12) Patent Application Publication
(10) Pub. No.: US 2008/0286812 A1
(43) Pub. Date: Nov. 20, 2008

(54) ALCOHOL OXIDASE-BASED ENZYME-LINKED IMMUNOSORBENT ASSAY

(76) Inventors: Joseph Thomas Ippoliti, Woodbury, MN (US); Katherine E. Olson, Lakeland, MN (US)

Correspondence Address: Intellectual Property Department;DEWITT ROSS & STEVENS S.C., Suite 600, 2 East Mifflin Street, Madison, WI, 53703-2865, US

(21) Appl. No.: 12/054,283

(22) Filed: Mar. 24, 2008

Related U.S. Application Data
(60) Provisional application No. 60/919,644, filed on Mar. 23, 2007.

(51) Int. Cl. G01N 33/53

(52) U.S. Cl. 435/7.9

(57) ABSTRACT

Disclosed is an assay for detecting an analyte. The method includes the steps of contacting a solution suspected of containing the analyte with capture antibodies specific for the analyte, wherein analyte contained in the solution is captured by the capture antibodies. Then contacting the capture antibodies with a solution containing the analyte attached to an alcohol oxidase (AOX) enzyme, to yield captured, labeled analyte. Then contacting the capture antibodies with a reagent mixture that generates a first signal proportional to the captured, labeled analyte and quantifying the first signal. And then measuring concentration of the analyte in the unknown sample by comparing the first signal to standard curve of signals. The assay can be implemented in an ELISA format.

### CROSS-REFERENCE TO RELATED APPLICATIONS

Priority is hereby claimed to provisional application Ser. No. 60/919,644, filed Mar. 23, 2007, which is incorporated herein by reference.

### BACKGROUND

The invention is directed to an enzyme-linked immunosorbent assay (an ELISA) that utilizes an alcohol oxidase enzyme and a hydrogen peroxide (H₂O₂)-sensitive latent fluorophore or latent chromophore to detect the presence and/or the amount of a pre-selected analyte in a sample (preferably a biological sample).

The ELISA format is widely utilized to assay for biologically active substances and need not be described in great detail here. By way of a brief summary, ELISA's utilize antigen-specific antibodies in concert with a specific antibody-enzyme conjugate to detect and quantify proteins, protein complexes and other antigens. The basic ELISA protocol can be modified in ways well known to the art to give different types of ELISA's, such as indirect, antibody-sandwich, and double antibody-sandwich ELISA's. By way of example, the basic protocol for a double antibody-sandwich ELISA is illustrated schematically in FIG. 1: A plate 12 is coated with antibodies 10 (called capture antibodies) specific for the analyte being assayed. The plate is then incubated with a blocking agent 14, such as bovine serum albumin (BSA) to block non-specific binding of proteins to the test plate. The test solution then is incubated on the plate coated with the capture antibodies, whereby the specific analyte being assayed 16 is “captured” from the test solution by the capture antibodies. The plate then is washed, incubated with specific detect antibodies 18, washed again, and incubated with a species-specific antibody-enzyme conjugate 20. After incubation, the unbound conjugate is washed from the plate and enzyme substrate is added 22. The presence of the bound antibody-enzyme conjugate results in a color change proportional to the amount of analyte which can be measured and quantified.

### SUMMARY OF THE INVENTION

A first version of the invention is directed to an assay for detecting an analyte. The method comprises a standard solution containing a known amount of unlabeled analyte, and providing a solution containing the analyte attached to an alcohol oxidase (AOX) enzyme to yield a labeled analyte. The standard solution is then contacted with capture antibodies specific for the analyte. The same capture antibodies are then contacted with the labeled analyte solution to yield capture antibodies having labeled analyte and unlabeled analyte attached thereto; and then contacting the capture antibodies with a reagent mixture that generates a first signal proportional to the captured, labeled analyte and quantifying the first signal; and repeating the steps using an unknown sample suspected of containing the analyte in place of the standard solution, to generate a second signal. The concentration of the analyte can then be measured in the unknown sample by comparing the second signal to the first signal.

It is preferred that the process be implemented in an ELISA format, in which case the capture antibodies are immobilized on a solid surface. It is preferred that the colorimetric reagent comprises a nascent fluorophore that is rendered fluorescent in the presence of H₂O₂, preferably peroxyfluor-1 or 2,2′-azino-bis(3-ethylbenzthiazoline-6-sulphonic acid (ABTS).

Another version of the invention is directed to an assay for detecting an analyte. Here, the method comprises contacting a solution suspected of containing the analyte with capture antibodies specific for the analyte, wherein analyte contained in the solution is captured by the capture antibodies; and then contacting the capture antibodies with a solution containing the analyte attached to an alcohol oxidase (AOX) enzyme, to yield captured, labeled analyte. The capture antibodies are then contacted with a reagent mixture that generates a first signal proportional to the captured, labeled analyte and quantifying the first signal. The concentration of the analyte in the unknown sample is then determined by comparing the first signal to standard curve of signals.

### BRIEF DESCRIPTION OF THE FIGURES

FIG. 1 is a schematic representation of a double antibody-sandwich enzyme-linked immunosorbent assay.

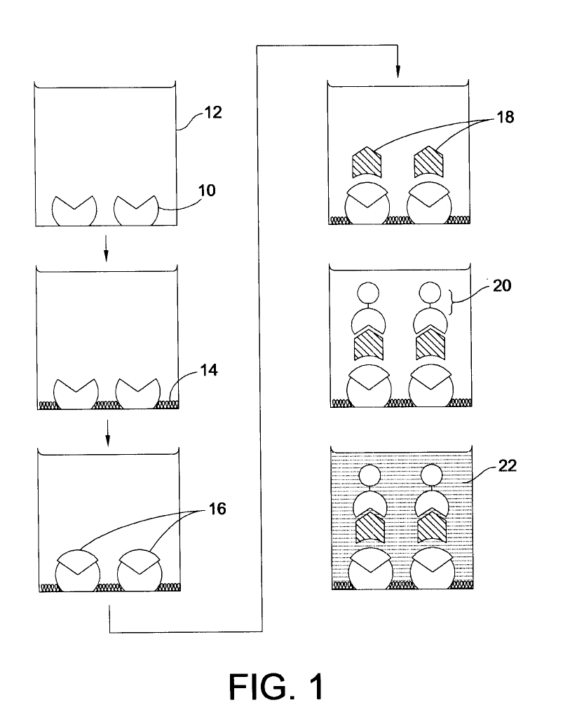

FIGS. 2A, 2B, 2C, and 2D together schematically depict one version of a competition ELISA according to the present invention. FIG. 2A shows capture antibodies 10 affixed to a solid support 12. FIG. 2B depicts analyte 16 being captured from solution by immobilized antibodies 10. FIG. 2C show adding labeled analyte 20, which binds to any remaining empty enzyme sites. FIG. 2D shows adding reagent 22 to induce a light-generating reaction that is proportional to the amount of labeled analyte 20 immobilized in each well. Each panel is shown in duplicate, with the left-hand panel in each figure having less analyte present, and the right-hand panel in each figure having more analyte present.

FIG. 3 is a logarithmic graph showing the optical density of ELISA tests when the concentration of estradiol (pM) is varied. As the concentration of estradiol was increased, the optical density decreased.

FIG. 4 is a graph depicting the change in fluorescent intensity over time for a solution containing 1 μM PF-1, 1:500 AOX, and 0.1% EtOH.

### DETAILED DESCRIPTION OF THE INVENTION

In the present invention, an alcohol oxidase (“AOX”) enzyme and a hydrogen peroxide-sensitive fluorophore (preferably Peroxyfluor-1, [“PF-1”]) or chromophore are utilized to make a sensitive enzyme-linked immunosorbent assay (ELISA) that can be used to test the concentration of biological species in solution by detecting the presence and/or concentration of hydrogen peroxide produced by AOX when exposed to alcohol. AOX (defined herein as any enzyme classified within E.C. 1.1.3.13) is an enzyme catalyzes the conversion of alcohols into aldehydes, thereby creating hydrogen peroxide as a by-product. (See U.S. Pat. No. 4,619,898, issued Oct. 28, 1986, to Hopkins entitled “Alcohol Oxidase from Pichia-type Yeasts,” which is incorporated herein by reference. The AOX enzyme described in this patent may be used in the present invention. Two latent chromophores are shown below.

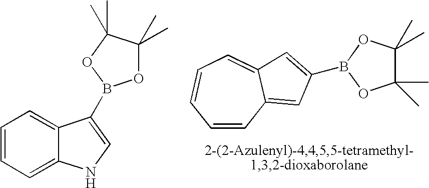

AOX can be isolated from the yeast Pichia pastoris, as well as a host of other species, by a number of method in addition to that described in U.S. Pat. No. 4,619,898. See, for example, Janssen, F. W. and Ruelius, H. W. “Alcohol oxidase, a flavoprotein from several Basidiomycetes species. Crystallization by fractional precipitation with polyethylene glycol,” Biochim. Biophys. Acta 151 (1968) 330-342; Nishida, A., Ishihara, T. and Hiroi, T. “Studies on enzymes related to lignan biodegradation,” Baiomasu Henkan Keikaku Kenkyu Hokoku (1987) 38-59 (in Japanese); Suye, S. “Purification and properties of alcohol oxidase from Candida methanosorbosa M-2003,” Curr. Microbiol. 34 (1997) 374-377. Alcohol oxidase from Pichia can also be purchased commercially from several suppliers, including Sigma-Aldrich (St. Louis, Mo.), Chematics (North Webster, Ind.), and Asahi Kasei Corporation (Tokyo, Japan).

Alcohol oxidase is produced by yeasts of the genus Pichia and yeasts that are genetically and/or taxonomically closely related to Pichia. These yeasts are generally capable of utilizing a feedstock containing methanol as a carbon and energy source. Specific examples of such methanol-utilizing Pichia yeasts that produce AOX include P. pastoris, P. pinus, P. trehalophila, and P. molischiana. Two exemplary strains of suitable yeasts of the species P. pastoris are available from the United States Department of Agriculture, Agriculture Research Service, Northern Regional Research Laboratories of Peoria, Ill., under the accession numbers NRRL Y-11430 and Y-11431.

To procure the AOX, a methanol-competent Pichia-type yeast is cultured under aerobic aqueous fermentation conditions using methanol as the carbon and energy source. Preferably the methanol is supplied under conditions so that methanol is the growth-limiting factor. The methanol limiting conditions are defined as a concentration of methanol which is the minimal concentration of methanol which results in a maximum growth rate for a given set of fermentation culture conditions. Preferably fermentation is conducted under high cell density conditions; cell density is preferably 100 grams or greater on a dry weight basis per liter of ferment. The selected yeast is grown in a batch or continuous process in the presence of oxygen, methanol, and an assimilable source of nitrogen. Various types of fermentation processes and apparatuses known in the art can be utilized. For example, a foam-type fermenter such as described in U.S. Pat. No. 3,982,998, or other suitable fermenter can be used.

Oxygen can be supplied to the fermenter as such, or in the form of air or oxygen-enriched air, in a range of pressures from such as about 0.1 atm. to 100 atm., as is known in the art. The assimilable source of nitrogen for the fermentation can be any organic or inorganic nitrogen-containing compound which provides nitrogen in a form suitable for metabolic utilization by the microorganisms. Suitable organic nitrogen sources include, for example, proteins, amino acids, urea, and the like. Suitable inorganic nitrogen sources include, for example, ammonia, ammonium hydroxide, ammonium nitrate, and the like. The presently preferred nitrogen sources include ammonia and ammonium hydroxide for convenience and availability.

The pH range in the aqueous microbial ferment should be in the range of about 3 to 7, more preferably and usually about 3.5 to 5.5. Preferences of certain microorganisms for a pH range are dependent to some extent on the medium employed, as well as on the particular microorganism, and thus may change somewhat with change in medium as can be readily determined by those skilled in the art.

Sufficient water is maintained in the fermentation means so as to provide for the particular requirements of the microorganism employed as well as to provide a carrier fluid for water soluble nutrients. Minerals, growth factors, vitamins, and the like, generally are added in amounts which vary according to the strain of microorganism utilized and the selected culture conditions, and are known to those skilled in the art or are readily determinable by them.

The growth of the microorganism is sensitive to the operating temperature of the fermenter and each particular strain of microorganism has an optimum temperature for growth. Exemplary fermentation temperatures are in the range of about 20° C. to about 65° C. The temperature selected will generally depend upon the microorganism employed in the process because each one will have a somewhat different temperature/growth rate relationship.

Fermentation pressures are generally within the range of about 0.1 to about 100 atmospheres, more usually about 1 to about 30 atmospheres, and more preferably about 1 to about 5 atmospheres. The higher pressures result in a greater level of dissolved oxygen in the aqueous medium and usually higher cell productivities.

To isolate the AOX, a fluid is prepared which is an aqueous suspension containing cells of the selected microorganism. The aqueous fluid can be fermenter effluent which can be used directly, or preferably after adjusting the pH as described below. Alternatively the suspended microorganism cells can be initially separated from the fermentation medium, for example, by centrifugation or by filtration through filters having a pore size less than the size of the individual cells, and subsequently resuspended in a convenient volume of water or of an appropriate aqueous buffer, for example KH₂PO₄/Na₂HPO₄ buffer at 0.2M. It has been found that the cell density in the aqueous suspension must be greater than a minimum crystallization density. Satisfactory results are obtained if the fluid cell density is greater than about 75 grams on a dry weight basis per liter of fluids. It has been found that satisfactory results are obtained if the fermenter effluent, where it is to be used as the fluid, is first adjusted to a pH of such as about 7.5 by addition of a base such as ammonium hydroxide, sodium hydroxide, and the like. The pH is not considered critical, however and the pH of the aqueous suspension need not be adjusted prior to homogenization. However, it is considered preferable to adjust the pH broadly in the range of about 6-9 since in this range the enzyme is active and stable.

The cell-containing fluid is homogenized by suitable means known in the art. For example, fermenter effluent containing yeast grown on methanol can be adjusted to a pH of about 7.5 and homogenized at a high cell density concentration such as 100-120 grams biomass (dry weight)/liter using a “DYNOMILL”-brand Model KDL homogenizer using a 0.6 liter vessel in a continuous operation at 5° to 30° C. using belt combination #3 and a flow of 20-30 ml/hr. The homogenate solids are separated from the homogenate to produce a crude solution containing alcohol oxidase as a soluble component. For example, the homogenate solids can be removed by centrifugation to yield a cell-free supernatant. Alternatively the solids can be removed by filtration through filters having a suitable pore size, followed by pH adjustment if desired. If desired, for further purification steps such as recovery of crystalline alcohol oxidase, the pH can be adjusted to have a pH in the range of 5.75 to 6.75 as desired, for example, to pH 6.5.

To purify the AOX, the crude solution containing the soluble alcohol oxidase can be treated to recover the AOX in more concentrated solid form by such as by fractional precipitation with ammonium sulfate, or by conventional dialysis modes or by applying ultrafiltration to increase the rate of recovery.

In dialysis, the crude solution containing the soluble AOX is dialyzed against a dialysis medium across a membrane impermeable to alcohol oxidase but permeable to water, buffer, and inorganic molecules. The crude solution is prepared by homogenizing an aqueous fluid having a cell density effective for crystallization of alcohol oxidase when the solution attains a recovery range solution condition as herein described. Satisfactory crystallization has been observed where the effective cell density is about 75 grams (on a dry weight basis) per liter of aqueous fluid. Crystallization is also expected to occur at even lower effective cell densities although the amount of crystalline alcohol oxidase recovered is less. Below an empirically determinable minimum cell density (minimum crystallization density) essentially no crystalline AOX is recovered. The type of membrane used is not considered critical and any suitable membrane may be used. For example, commercially available cellulose acetate dialysis tubing can be used to form dialysis bags or otherwise used, or hollow fiber dialysis cells can be used. The alcohol oxidase containing solution is dialyzed against a dialysis medium, for example water or a buffer solution, to achieve a recovery range solution on the enzyme side of the membrane having an ionic strength in a recovery range of between 0.05M and 0.01M thereby effecting precipitation of an electrophoretically homogeneous crystalline oxidase. The dialysis medium can be any medium whereby during dialysis the molar ionic strength of the solution on the enzyme side of the membrane passes through at least a portion of the recovery range. For example, if the crude solution containing alcohol oxidase has a molar ionic strength of 0.2M, the dialysis medium can be a suitable volume of distilled water. The volume of fluid against which the enzyme is dialyzed is not considered critical so long as the ionic strength on the enzyme side of the membrane passes through at least a portion of the recovery range.

During dialysis, the pH of the alcohol oxidase containing solution should be maintained in the range of about 5.75 to about 6.75 by use of a suitable buffer system. A suitable buffer system comprises, for example, potassium dihydrogen phosphate and disodium hydrogen phosphate. Preferably the pH range is from about 6.0 to about 6.5 for recovery of maximum amounts of crystalline AOX. Good crystallization of the AOX has been observed within the broad pH range.

At the end of dialysis, the AOX is present in the dialysis bag as a crystalline solid. The crystalline alcohol oxidase can be readily separated from the dialysis medium, such as by decanting the liquid in the dialysis bag from the solid crystals. The moist crystals can be further processed as desired for storage. For example, the crystal slurry can be frozen followed by lyophilization to form a dry powder, or can be dissolved in water or more preferably in a phosphate buffer. Stabilizer compounds known to stabilize enzyme solutions against denaturation and loss of enzymatic activity can be added, such as surcrose or glycerol. It is preferable to store the prepared enzyme at temperatures in the range of about 4° C. to 40° C. Only minimal loss of activity has been found to occur when the enzyme is stored at 4° C. in 0.1M phosphate buffer at pH 7.5, and with 0.02% sodium azide to inhibit microorganism growth. The AOX can also be stored frozen without significant loss of enzymatic activity.

In the present invention, the hydrogen peroxide liberated by the action of an AOX is used to convert a latent, hydrogen peroxide-sensitive fluorophore into an active fluorophore. Thus, when incorporated into an ELISA format, the hydrogen peroxide formed by the action of AOX in turn generates a proportional amount of active fluorophore from the latent fluorphore. The amount of the liberated fluorophore can then be measured by conventional means using conventional fluorescence-measuring equipment to determine the amount and/or presence of an analyte in a sample. The preferred latent fluorphore is PF-1, which is converted by the action of hydrogen peroxide into fluorescein, a well-known and highly fluorescent chemical. Chang, M. A. Pralle, E. Isacoff, and C. Chang (2004) “A selective, cell permeable optical probe for hydrogen peroxide in living cells,” J. Amer. Chem. Soc. 126:15392-15393.

Thus, in the present invention, an ELISA, such as a competition ELISA or any other ELISA-type format, is arranged between a known amount of the desired analyte, linked to an AOX, and the same analyte present in a sample to be tested. In short, in the preferred version of the invention, a standard curve is established using known quantities of the analyte to be measured, in an ELISA format that utilizes an AOX enzyme. The standard curve can then be used to determine the presence and amount of the same analyte in an unknown sample. The analyte can be literally any compound, without limitation, i.e., proteins, steroid, hormones, etc., for example, estradiol, testosterone, progesterone, and the like, as long as it can be chemically linked to AOX to make an AOX-analyte conjugate complex.

The change in fluorescence between the standards used to establish the standard curve, and the test sample, is then quantified to determine the concentration of the analyte in the unknown test sample. In the context of a competitive ELISA, a low quantifiable change in fluorescence correlates with a high concentration in the sample because the analyte in the sample out-competes the AOX-analyte conjugate. Quantifying these unknown samples can be used to test for varied health conditions associated with changes in hormone levels, or in levels of other biologically important compounds.

The primary benefit of the present invention is that it is both very sensitive and very robust. Additionally, the AOX enzyme itself is very robust and inexpensive. The assay method described herein does not require any additional enzymes to function—thus it is highly cost-effective. It is very easy to attach the AOX enzyme to various analytes. And, by using a single substrate and a novel indicator, the assay is both very simple to use and highly sensitive. The AOX enzyme is also stable under different pH ranges that alkaline phosphatases and other common enzymes used in the ELISA format are not. Thus, the present assay can be used under conditions where an alkaline phosphatase ELISA may not function optimally. Also, any hydrogen peroxide sensitive chromophore can be used to detect the presence of the AOX enzyme.

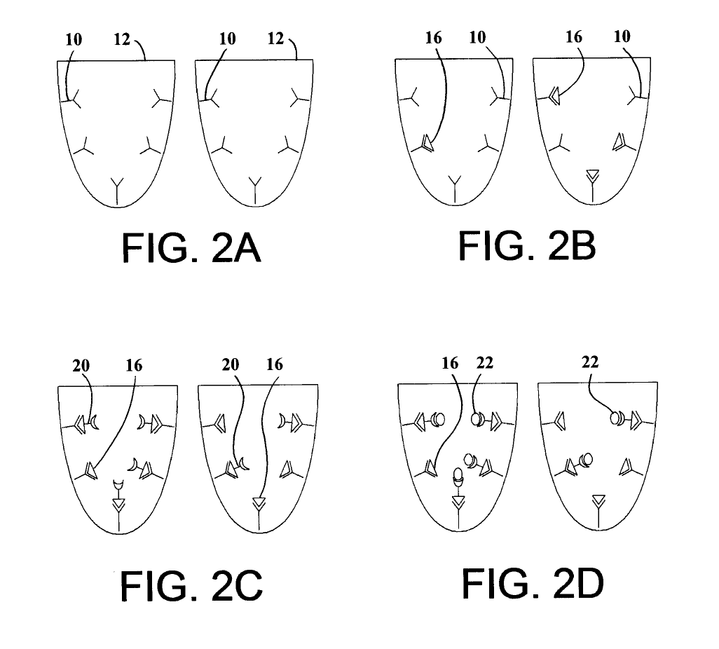

The preferred ELISA process of the present invention, for two different samples (left and right wells) is shown schematically in FIGS. 2A, 2B, 2C, and 2D. The preferred version of the ELISA according to the present invention comprises attaching capture enzymes 10 to a solid support in a well 12, as shown in FIG. 2A. As shown in FIG. 2A, the antibodies 10 are exposed to control solutions containing known concentrations of the real antigens 16 to be detected. The antigens 16 are captured from solution by the antibodies 10. The sample on the left in FIG. 2B has a smaller number of antigens than the sample on the right in FIG. 2B. As a result, fewer antigens 16 are bound in the left-hand panel in FIG. 2B as compared to the right-hand panel in FIG. 2B. Any non-bound, free antigens are then washed away. (Not shown.) Then antigens with a luminescent moiety 20 (i.e., labeled antigens) are added to the wells as shown in FIG. 2C. The uncomplexed or empty antibodies will complex with, and capture, the labeled antigens 20 from solution. The wells are again washed to remove excess labeled antigen (not shown). Then, as shown in FIG. 2D, appropriate activating chemicals 22 are added and the light intensity from each well is measured. The light from these “labeled” antigens is be proportional to the number of empty antibody sites that were present in FIG. 2B. This enables the amount of antigen that is actually present in each sample to be calculated by difference in an inverse relationship—the more light detected, the less antigen was initially present. These values can be quantified by comparison to standard samples (i.e., by comparison to a standard curve of light values).

### EXAMPLES

### Example 1

### E2-AOX Preparation

Estradiol-3-carboxymethyl ether was prepared with estradiol and sodium chloroacetate that underwent an SN2 reaction using 50% NaOH to deprotonate the phenolic hydrogen. See Reaction Scheme 1. After synthesizing the estradiol-3 carboxymethylether it was activated through a series of reactions, giving it the ability to react with the lysine side chains of the AOX. See Reaction Scheme 2. The activated form was then combined with AOX and allowed to react at room temperature. See Reaction Scheme 3. The resulting conjugate was then washed to remove any unreacted AOX and estradiol starting products. See Hermanson, Greg T. “Bioconjugate Techniques,” San Diego: Academic Press, 1996. 139-140, 630-633.

### Synthesis and Activation of Estradiol-3 Carboxymethylether

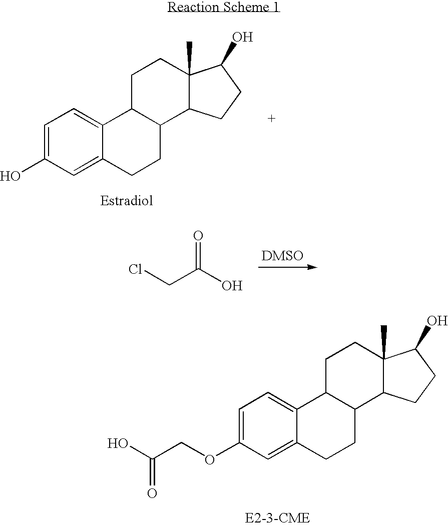

### Activation of Estradiol-3 Carboxymethylether

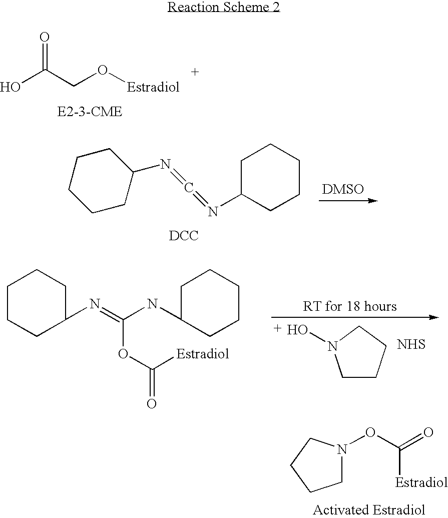

### Reaction of Estradiol-3 carboxymethylether with AOX

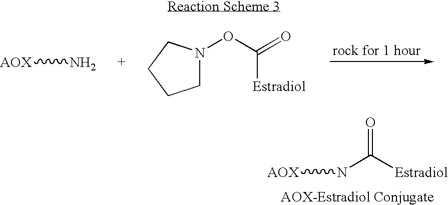

To ensure that binding between the activated estradiol and AOX has taken place, the extinction coefficients were calculated at 280 nm. The AOX-Estradiol conjugates showed higher extinction coefficients than AOX. This makes sense because in the AOX-E2 conjugate, both AOX and estradiol are contributing to the absorbance. See Table 1.

TABLE 1
Calculated extinction coefficients (M⁻¹ cm⁻¹⁾ for AOX and AOX-Estradiol
(AOX-E2) with varying equivalents of E2-NHS.

| | AOX-E2 | AOX-E2 | AOX-E2 |
|---|---|---|---|
| AOX | (10) | (30) | (50) |
| 828 | 1174 | 1224 | 2746 |

### Synthetic Materials and Methods.

Peroxy Crimson 1 (PC1) was synthesized according to literature procedures with some modification where noted. See Chang, C. et al. Molecular imaging of hydrogen peroxide produced for cell signaling. Nature Chemical Biology, 3: 5, 263-267 (2007). All chemicals were purchased from Sigma-Aldrich (St. Louis, Mo.) and were used as received. ¹H NMR spectra were collected in CDCl₃ at 25° C. on a Bruker AV-300 spectrometer at the University of St. Thomas, Chemistry Department in St. Paul, Minn.

### 3-Oxo-3H-phenoxazin-7-yl trifluoromethanesulfonate

Resorufin sodium salt (1, 516.6 mg, 2.2 mmol) and N-phenyl bis(trifluoromethanesulfonamide) (787.0 mg, 2.2 mmol) were dissolved in 40 mL of dry DMF in a 100 mL RB flask. N,N-diisopropylamine (Hunig's base, 1.1 mL, 6.6 mmol) was added via syringe, and the resulting solution was stirred at room temperature, in the dark for 24 hours. The reaction mixture was crashed into 300 mL of chilled brine and filtered through a glass frit which delivered product 2 as a yellow product (721 mg, 95%). ¹H NMR (CDCL₃, 300 MHz): δ 7.9 (1 h, d, J=9.6 Hz), 7.45 (1H, d, J=10 Hz), 7.29 (2H), 6.90 (1H, dd, J₁=9.8 Hz, J₂=1.8 Hz), 6.37 (1H, d, J=2.0 Hz).

### 3-Oxo-3H-phenoxazin-7-yl pinacolatoboron (Peroxy Crimson 1, PC1)

To a 50 mL RB flask, bis(pinacolato)diboron (140 mg, 0.5525 mmol), PdCl₂(dppf) CH₂Cl₂ (44 mg, 0.0533 mmol), potassium acetate (142 mg, 1.455 mmol), and 5 (171 mg, 0.4962 mmol) were added. To this mixture 30 mL of anhydrous THF was canulated into the RB. Nitrogen was bubbled through this reaction mixture for 30 minutes, upon which the RB was quickly placed onto the bottom of a reflux condenser, under positive nitrogen pressure. The reaction was heated to 80° C. overnight using an oil bath and the J-Kem Scientific. The reaction was then cooled to room temperature, diluted with 60 mL of toluene, and filtered in a D size glass frit over a pad of celite. The organic layer was washed with brine (3×100 mL) using a separation funnel and dried over 5 grams of MgSO₄. The solvent was then removed in vacuo to leave a brown/brick red residue. The residue was washed with 10 mL of chilled methanol to provide PC1 (3) as a brick red solid (13 mg, 8% yield). ¹H NMR (CDCl₃, 300 MHz): σ 7.75 (3H, m), 7.4 (1H, d), 6.9 (1H, d), 6.3 (1H, d), 1.4 (12H, s).

3-Bromoindole: A solution containing liquid bromine (20.857 mmol) in DMF (35 mL) was added drop wise via liquid addition funnel to a solution containing indole (20.857 mmol) dissolved in DMF (35 mL). The reaction was allowed to stir in the dark for 40 minutes. After the stirring the reaction was poured into a 10M potassium bisulfite solution containing 0.5% ammonium hydroxide. The solution was then stirred and allowed to sit for 5 minutes. The solution was then filtered with a C sized frit and a light white solid was collected. The solid was either immediately reacted or stored using a high vacuum in the dark. If the solid was not reacted or stored under vacuum then it quickly oxidized overnight and became unusable.

Borate ester of indole: The formation of the borate ester at the 3 position of indole to form the borate ester of indole proved to be more difficult. Four different reaction conditions were tried however a satisfactory method of purification was not found.

Reaction Conditions 1: 3-bromoindole (5.319 mmol), bis(pinacolato)diboron (7.323 mmol), [1,1′-Bis(diphenylphosphino)-ferrocene]dichloropalladium(II) Pd complex with dichloromethane (0.5689 mmol), and potassium acetate (3.391 mmol) were added to THF (15 mL) in a microwave vial with a nitrogen atmosphere. These reagents were microwaved for 5 minutes at 150° C. The reaction mixture was then purified by filtration through silica gel and washing it with 500 mL ethyl acetate/hexanes (95/5). The solution was then rotary evaporated to a solid. NMR analysis of the rotary evaporated solution revealed that the desired product was not obtained in high yield. In addition there were many impurities within the crude product.

Reaction Conditions 2: The second conditions 3-bromoindole (1.045 mmol), bis(pinacolato)diboron (3.083 mmol), [1,1′-Bis(diphenylphosphino)-ferrocene]dichloropalladium(II) Pd complex with dichloromethane (0.0338 mmol), and potassium acetate (6.040 mmol) were added to DMF (10 mL) in a microwave vial with argon atmosphere. The vial was microwaved for 5 minutes at 150° C. The reaction mixture was poured into 70-mL of deionized water and a brown solid immediately crashed out. The brown solid was re-dissolved in methylene chloride and rotary evaporated dry. The product was dissolved in methylene chloride/methanol (99:1) and the re-dissolved product was purified using flash chromatography (methylene chloride/methanol 99:1). Two solids and one residue were obtained from the 60 fractions taken. The first solid was a light green solid which appeared to be oxidized starting material specifically 3-bromoindole. The second solid was a light pink yellow solid, which by NMR analysis appeared to be the desired product. The solid was tested alone with hydrogen peroxide and dissolved in DMF with hydrogen peroxide but failed to give a color change under either condition, The dark brown residue attained form the fractions was determined by NMR analysis to be a mixture of solvents.

Reaction Conditions 3: In a nitrogen glove box, 3-bromoindole (5.2127 mmol), bis(pinacolato)diboron (15.740 mmol), [1,1′-Bis(diphenylphosphino)-ferrocene]dichloropalladium(II) Pd complex with dichloromethane (1.0986 mmol), and potassium acetate (40.730 mmol) were added to a microwave vial and sealed. Anhydrous DMF (10 mL) was added. The reaction mixture was heated to 120° C. for 5 minutes however the vial had to be vented because of high pressure. The reaction mixture was poured into 100-mL of deionized water and a black solid immediately crashed out. The solid was collected by filtration and re-dissolved in 100 mL of methylene chloride. 100 mL of hexanes was then added and a black solid crashed out of solution. An NMR of both the filtrate and the filtered solid were taken. The filtrate did not contain the desired product. The black solid that was collected by filtration likely contained the product however it could not be dissolved in methylene chloride, hexanes or ethyl acetate.

Reaction Conditions 4: 3-bromoindole (3.0147 mmol), bis(pinacolato)diboron (3.0876 mmol), [1,1′-Bis(diphenylphosphino)-ferrocene]dichloropalladium(II) Pd complex with dichloromethane (0.2781 mmol), and potassium acetate (0.8345 mmol) were refluxed in THF (50 mL) for 24 hours. The reaction was quenched with water and then extracted with ethyl acetate. The reaction mixture containing the product then needed to be purified using flash chromatography however an appropriate solvent system has not yet been found. 100% dichloromethane left the TLC plates streaky. Varying the concentration of methanol still left streaky TLC plates. Spotting the 3-bromoindole under the same conditions left streaks as well. An appropriate solvent system is still being explored.

Chromophore-3: 2-(2-Azulenyl)-4,4,5,5-tetramethyl-1,3,2-dioxaborolane: Bis(pinacolato)diboron (0.5 mmol), 2,2′-bipyridine (0.05 mmol), chloro(1,5-cyclooctadiene)iridium(t) dimer (0.025 mmol), and azulene (1.1 mmol) were added to a solution of dry cyclohexane (25 mL). The mixture was refluxed for 17 hours with a nitrogen atmosphere. The reaction mixture was concentrated by rotary evaporation to a dark blue oily residue and then purified by flash column chromatography(silica; hexane/ethyl acetate 5:1). (Kuritibi, Kei et al. 2003. Direct Introduction of a Boryl Substituent into the 2-position of Azulene: Application of the Miyaura and Smith Methods to Azulene. European Journal of Organic Chemistry, 3663-3665.) NMR analysis revealed that fraction 7 contained a mixture of the isomers of Chromophore-3,2-(2-Azulenyl)-4,4,5,5-tetramethyl-1,3,2-dioxaborolane and its isomer 2-(1-Azulenyl)-4,4,5,5-tetramethyl-1,3,2-dioxaborolane. The mixture of isomers was a dark purple solid. Other fractions contained unreacted starting material and catalysts.

Qualitative Testing of 2-(2-Azulenyl)-4,4,5,5-tetramethyl-1,3,2-dioxaborolane and 2-(1-Azulenyl)-4,4,5,5-tetramethyl-1,3,2-dioxaborolane with hydrogen peroxide: The mixture of isomers was tested for its ability to change color in the presence of hydrogen peroxide. A few milliliters of methanol were added to a few milligrams of the mixture of isomers. The methanol-isomer solution was violet in color. A few drops of 30% hydrogen peroxide were added to the solution. The solution immediately began changing color to a dark red color. After approximately 20 minutes the color had completely changed to a dark red.

### Example 3

### ELISA Procedure

A protocol was developed to ensure the activity of the AOX and the binding of the E2 to the antibody. This was a modification of the procedures given in Crowther, John R. “The ELISA Guidebook,” Totowa, N.J.: Humana Press, 2001. 9-14. Sensitivity was then determined using the calorimetric assay.

1. Coat wells with antibody using coating buffer. Wash 3×. Add blocking buffer to prevent E2 and AOX from binding non-specifically to the surface of the well. Wash 3×.

2. Add estradiol samples (the exemplary analyte in this example) to the wells. Sample A has a lower concentration of E2 than sample B.

3. Add E2-AOX conjugates to the wells. Wash 3×.

4. Add substrate specific for AOX (2,2′-azino-bis(3-ethylbenzthiazoline-6-sulphonic acid (i.e., ABTS) or PF-1). Measure color change at 450 nm using a spectrophotometer.

These steps are illustrated schematically in FIGS. 2A through 2D and described above. The change in absorbance is inversely proportional to the concentration of unbound E2.

### Example 4

### ELISA Sensitivity

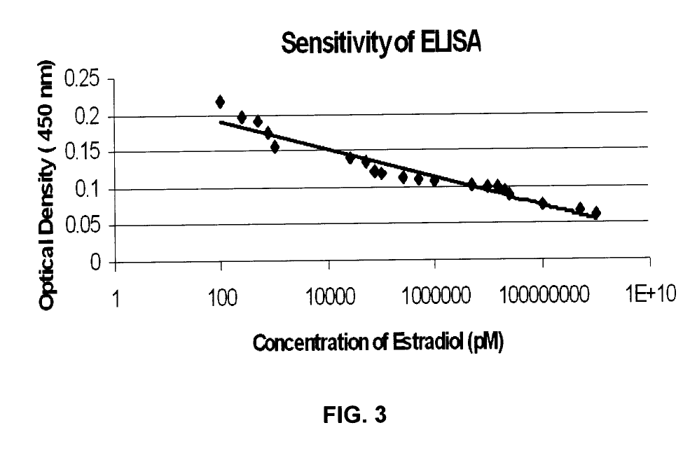

Tests were performed to determine the sensitivity of the ELISA for varying concentrations of estradiol. These tests showed a sensitivity up to about 100 μM. See FIG. 3, which is a graph depicting the results for varying concentrations of estradiol. FIG. 3 is a logarithmic graph showing the optical density of ELISA tests when the concentration of estradiol (pM) is varied. As the concentration of estradiol was increased, the optical density decreased. These results are significant because they show that the ELISA according to the present invention gives linear results the correlate with concentration over several order of magnitude concentration of analyte

### Example 5

### Synthesis of PF-1

Reaction Scheme 4a depicts the Reaction of 3-iodophenol with phthalic anhydride to yield a 3,6-diiodofluoran. Reaction Scheme 4b shows the reaction of the 3,6-diiodofluoran with bis(pinacolato)diboran.

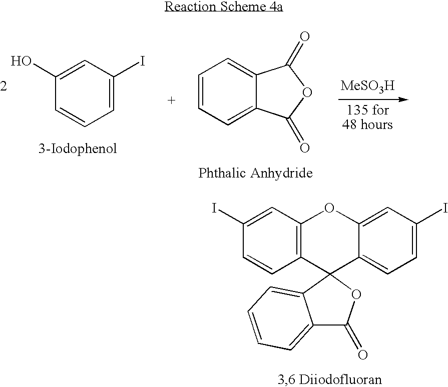

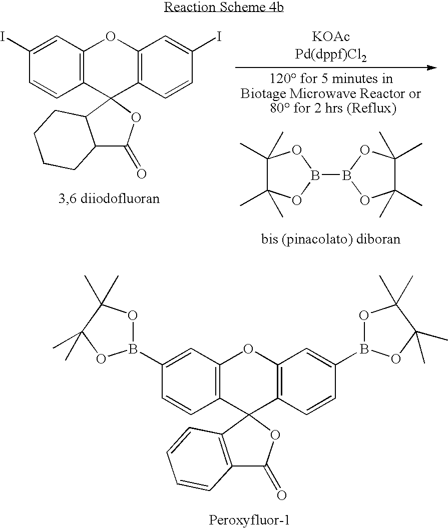

Reaction Scheme 5 depicts the reaction of PF-1 with H₂O₂. The reaction is catalyzed by AOX. This reaction yields the fluorescent compound fluorescein. In short, AOX creates H₂O₂ by converting an alcohol to an aldehyde. The H₂O₂ then reacts with the PF-1 to create fluorescein:

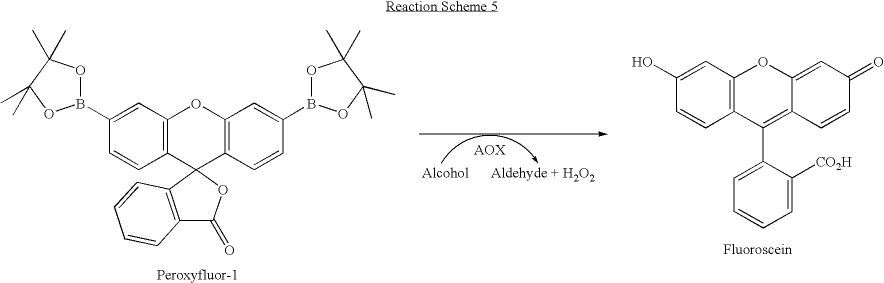

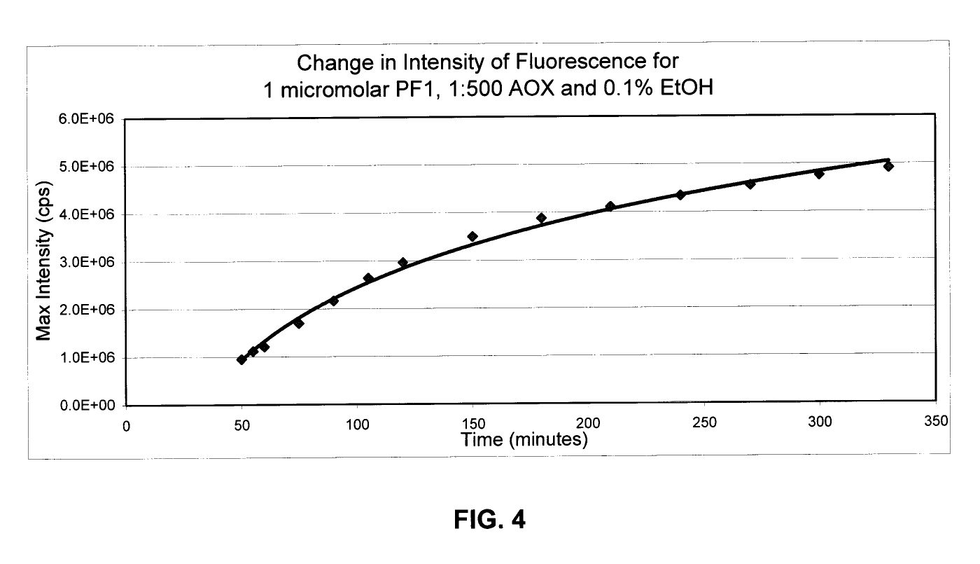

The production of fluoroscein from the PF-1 is proportional to amount of H₂O₂ produced, which in turn is proportional to the amount of AOX. Thus, the latent fluorophore, which is activiate by the present of H₂O₂ is perfect for an assay for detecting AOX. This utility is depicted in FIG. 4. FIG. 4 is a graph depicting the change in fluorescent intensity over time for a solution containing 1 μM PF-1, 1:500 AOX, and 0.1% EtOH. As can be seen in the graph, the PF-1 is highly sensitive to the presence of H₂O₂ which is generated by the action of the AOX on the ethanol.

### Example 6

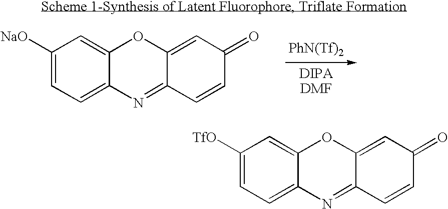

Treatment of Resorufin Sodium Salt with N-phenyl bis(trifluoromethanesulfonamide) gives a triflate leaving group. DIPA, (N,N-diisopropylamine), in DMF, dark, RT,

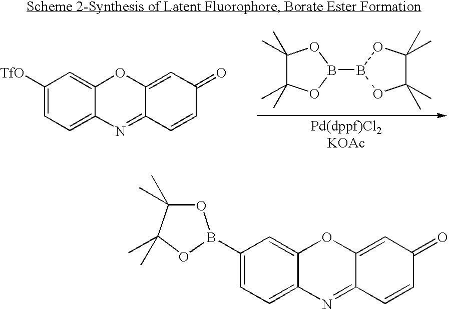

THF, 80° C., nitrogen atmosphere, Bis(pinacolato)diboron, KOAc, (Potassium Acetate)

### Example 7

Latent chromophore 2 and 3 can be used with an AOX linked antigen in an ELISA to undergo the following reactions that produce color changes.

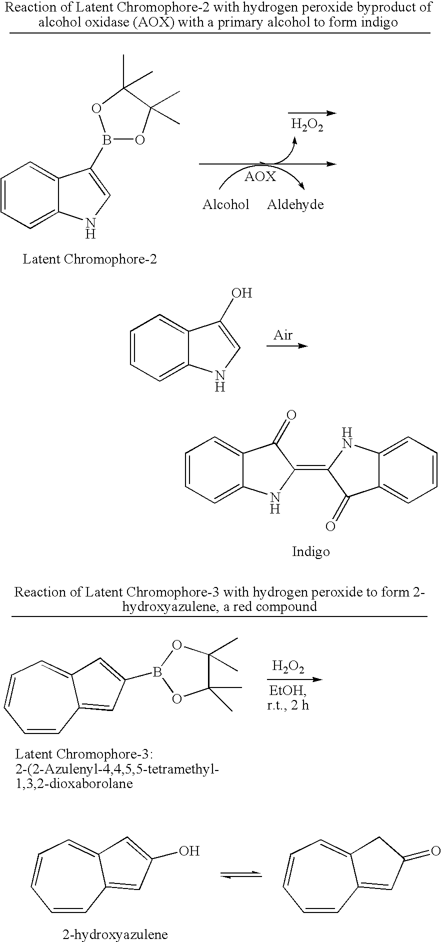

What is claimed is:

1. An assay for detecting an analyte comprising:
(a) providing a standard solution containing a known amount of unlabeled analyte, and
(b) providing a solution containing the analyte attached to an alcohol oxidase
(AOX) enzyme to yield a labeled analyte; and then
(c) contacting the standard solution of step (a) with capture antibodies specific for the analyte, and then contacting the same capture antibodies with the solution of step (b), to yield capture antibodies have labeled analyte and unlabeled analyte attached thereto; and then
(d) contacting the capture antibodies of step (c) with a reagent mixture that generates a first signal proportional to the captured, labeled analyte and quantifying the first signal; and
(e) repeating steps (c) and (d) using an unknown sample suspected of containing the analyte in place of the standard solution, to generate a second signal; and then
(f) measuring concentration of the analyte in the unknown sample by comparing the second signal to the first signal.

2. The assay of claim 1, wherein the capture antibodies are immobilized on a solid surface.

3. The assay of claim 1, wherein the AOX enzyme is isolated from a yeast of the genus Pichia.

4. The assay of claim 1, wherein the reagent of step (d) comprises a latent fluorophore that is rendered fluorescent in the presence of H₂O₂.

5. The assay of claim 1, wherein the reagent of step (d) comprises peroxyfluor-1.

6. The assay of claim 1, wherein the reagent of step (d) comprises 2,2′-azino-bis(3-ethylbenzthiazoline-6-sulphonic acid (ABTS).

7. The assay of claim 1 wherein the reagent of step (d) comprises latent chromophore 2 shown below.

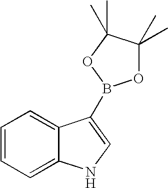

8. The assay of claim 1, wherein the reagent of step (d) comprises 2-(2-Azulenyl)-4,4,5,5-tetramethyl-1,3,2-dioxaborolane (latent chromophore 3) shown below.

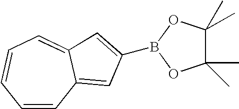

9. An assay for detecting an analyte comprising:
(a) contacting a solution suspected of containing the analyte with capture antibodies specific for the analyte, wherein analyte contained in the solution is captured by the capture antibodies; then
(b) contacting the capture antibodies of step (a) with a solution containing the analyte attached to an alcohol oxidase (AOX) enzyme, to yield captured, labeled analyte; and then
(c) contacting the capture antibodies of step (b) with a reagent mixture that generates a first chemiluminescent signal proportional to the captured, labeled analyte and quantifying the first signal; and
(d) measuring concentration of the analyte in the unknown sample by comparing the first signal to standard curve of signals.

10. The assay of claim 9, wherein the capture antibodies are immobilized on a solid surface.

11. The assay of claim 9, wherein the AOX enzyme is isolated from a yeast of the genus Pichia.

12. The assay of claim 9, wherein the reagent of step (c) comprises a nascent fluorophore that is rendered fluorescent in the presence of H₂O₂.

13. The assay of claim 9, wherein the reagent of step (c) comprises peroxyfluor-1.

14. The assay of claim 9, wherein the reagent of step (c) comprises 2,2′-azino-bis(3-ethylbenzthiazoline-6-sulphonic acid (ABTS).

15. An assay for detecting an analyte comprising:
(a) providing a standard solution containing a known amount of unlabeled analyte, and
(b) providing a solution containing the analyte attached to an alcohol oxidase
(AOX) enzyme to yield a labeled analyte; and then
(c) contacting the standard solution of step (a) with capture antibodies, and then contacting the same capture antibodies with the solution of step (b), to yield capture antibodies have labeled analyte and unlabeled analyte attached thereto; and then
(d) contacting the capture antibodies of step (c) with a reagent mixture comprising PF-1 or ABTS, where the reagent mixture generates a first chemiluminescent signal proportional to the captured, labeled analyte and quantifying the first signal; and
(e) repeating steps (c) and (d) using an unknown sample suspected of containing the analyte in place of the standard solution, to generate a second signal; and then
(f) measuring concentration of the analyte in the unknown sample by comparing the second signal to the first signal.
